(19) United States
(12) Patent Application Publication
(10) Pub. No.: US 2007/0077435 A1
(43) Pub. Date: Apr. 5, 2007

(54) Process for coating a medical device

(76) Inventors: Deborah M. Schachter, Edison, NJ (US); Raymond S. Shissias, Iselin, NJ (US); Caner Umit Yurteri, Gainsville, FL (US); Eduardo C. Escallon, Elwood, IN (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/243,648

(22) Filed: Oct. 5, 2005

(51) Int. Cl. A61L 33/00; B32B 9/04

(52) U.S. Cl. 428/411.1; 427/2.1

(57) ABSTRACT

An improved process for electrostatically coating a medical device is described. Temporary conductivity is induced into the surface of a medical device whose surface is normally non-conductive. After inducing temporary conductivity to the surface of the device, it is electrostatically coated either through liquid formulation spray coating or through dry powder deposition. The process provides a high degree of uniformity and control over the coating such that the fine features of the device that are necessary for device function are carefully maintained after the coating is applied.

### FIELD OF THE INVENTION

The present invention relates to an improved process for coating a non-conductive medical device. Specifically, this invention relates to an improved process for electrostatically spray coating a non-conductive medical device with an electrically charged coating formulation.

### BACKGROUND OF THE INVENTION

Coatings for medical devices serve a myriad of useful purposes. For example, coatings can be used to change device surface properties, to incorporate drug/bioactive or antimicrobial agents for release from the surface of the device, or to provide for cell signaling for better healing. However, oftentimes the medical device is in the form of a tissue engineering scaffold or a device with complex architecture. In both cases, these devices require fine coatings that closely follow the micro-scale detail of the device. Coatings of this quality are not easily achievable with traditional dip or spray coating. In addition, the coating materials can be exceedingly expensive if they contain drugs or bioactives and therefore the waste that is generated with these methods renders these processes prohibitive for use in many medical device based applications.

In contrast, electrostatic deposition processing is a highly controllable method that provides for coatings that track the detail and architecture of the substrate. Due to the targeted nature of the electrostatic deposition process, there is very little overspray or waste associated with it. Targeting is the result of the attraction between charged particles and grounded substrate. The limitation of electrostatic deposition lies in the types of substrate that can be coated using this method. Electrostatic deposition requires that the substrate be conductive. Conductivity allows the substrate to be grounded and thereby attract coating particles. It also provides for the relaxation of the charge on the coating particle, converting it into a micro-current and thereby maintaining the particles on the surface.

Electrostatic coating methods have been suggested for coating medical devices. For instance, U.S. Pat. Nos. 6,355,058, 5,824,049 and 6,096,070 mention the use of electrostatic deposition to coat a medical device with a radiopaque or bioactive material. In the conventional electrodepositing or electrostatic spraying method, the surface of the medical device is grounded and a gas is used to atomize the coating solution into droplets. The droplets are then electrically charged using, for example corona discharge. The gas-atomized droplets are electrically charged by passing through a corona field. Since the droplets are charged, they are attracted to the grounded surface of the device.

The electrostatic coating of medical devices was also suggested in U.S. Pat. No. 6,669,980. In the method described in this patent, the coating formulation is charged in a specific nozzle that causes the liquid jet to break up into a spray cone of highly charged droplets due to charge repulsion between droplets, consequently eliminating the need for gas atomization. The '980 patent described the uniform and even coating on a conductive medical device such as a metallic stent. It further suggests that this method would also be appropriate for polymeric-based medical devices. However, the static charge accumulated on such devices results in the repulsion of the coating particles by the device, thus leading to undesirable results.

In view of the deficiencies of the prior art, there is a need for an improved coating process for electrostatically coating a medical device, particularly when the surface of the device is a non-conductive surface such as those surfaces fabricated from polymeric materials. Significantly, an improved process is needed which will avoid the inevitable static charge build-up during electrostatic spraying or deposition that actually will repel coating particles, thus leading to undesirable coating results.

### SUMMARY OF THE INVENTION

The present invention is an improvement to the known process for the electrostatic coating of a medical device. In that process, a medical device is first provided. The device is placed on a metallic support, and the device is grounded. The surface of the grounded medical device is then electrostatically coated with a coating formulation.

In the improved process of this invention, the medical device has a surface that is non-conductive. The improvement comprises inducing a temporary conductive layer on the non-conductive surface of the medical device prior to the step of electrostatic coating of the surface of the device with the coating formulation. In the preferred embodiment, the temporary conductive layer is induced using a polar solvent.

Advantageously, the inducement of a temporary conductive layer on the surface of the medical device prior to electrostatic coating properly grounds the device so as to relax any static charge build up. Consequently, the surface of the medical device attracts the electrostatically charged coating rather than repel it. In this way, a desirable coating can be applied to the surface of the medical device and the process parameters can be carefully controlled to provide a high degree of uniformity such that the fine features of the device that are necessary for device function can be carefully maintained after the coating is applied.

The improved coating process of this invention can be used to coat the surfaces of numerous medical devices such as tissue engineering scaffolds and complex-shaped medical devices such as bone screws.

### BRIEF DESCRIPTION OF THE FIGURES

FIG. 1: A scanning electron micrograph (SEM) of a cross-section of a non-conductive non-woven scaffold electrostatically coated with a liquid coating formulation in accordance with the prior art without pre-treatment of the scaffold with a polar solvent to induce a temporary conductive layer.

FIG. 2: A scanning electron micrograph (SEM) of a cross-section of a non-conductive non-woven scaffold electrostatically coated with a liquid coating formulation with pre-treatment of the scaffold with a polar solvent to induce a temporary conductive layer.

### DETAILED DESCRIPTION OF THE INVENTION

In the improved coating process of the present invention, a medical device having a non-conductive surface is provided. The device is placed on a metallic support, and then grounded. Importantly, and in accordance with the improved process of this invention, temporary conductivity is induced onto the surface of the device. Ideally, temporary conductivity is induced by either dipping the device into, or spraying the device with, a polar liquid.

A suitable polar solvent for use in the present invention is one that can “wet” the device and induce temporary conductivity to the device without dissolving or damaging the device in any way. The length of time that the device remains conductive is dependent upon the volatility of the polar liquid. A less volatile polar solvent such as N-methylpyrrolidone would allow for longer coating times than a more volatile polar solvent such as isopropanol. Examples of polar liquids for use in the present invention include but are not limited to tetrahydrofuran (THF), acetone, ethyl acetate, N-methylpyrrolidone (NMP), dimethyl sulfoxide (DMSO), alcohols such as isopropanol or ethyl alcohol, methylene chloride, methyl ethyl ketone (MEK), and mixtures thereof. Ethyl acetate and isopropanol are the preferred polar solvents.

Once the temporary conductive layer on the surface of the medical device is induced, the device may then be effectively coated electrostatically with a coating formulation. The coating method is dependant upon the form of the coating formulation and the complexity of the medical device. Forms of coating formulations include liquids, such as solutions of polymers in solvents, or polymers in the form of emulsions or suspensions. Alternatively, powders such as monomer or polymer powders can be used.

In one embodiment, the coating formulation is a liquid, such as a solution of polymer in a solvent, in an emulsion, or in a suspension. The liquid coating formulation is electrostatically applied as described, for example, in U.S. Pat. No. 4,749,125. In the case of complex-shaped medical devices, it is preferable that an inductor ring is centered around the nozzle tip instead of a conductor. An inductor ring is similar to an inductor bar described in U.S. Pat. No. 5,332,154. The inductor ring is either grounded or held at some voltage level lower than the voltage at the nozzle itself. The droplets of the electrically charged coating formulation created are dispensed through the nozzle opening, flow through the inductor ring, and then are deposited on the grounded complex-shaped medical device surface.

Although the nozzle apparatus can be made of any insulative material, such as polyamide, preferably, it is made of ceramics. Also, preferably, the flow rate of the coating formulation at the opening of the nozzle apparatus is at about 0.1 milliliter per hour (ml/hour) to about 10 ml/hour. Additionally, the amount of voltage used to charge the coating formulation preferably ranges from about 4 kilovolts (kV) to about 20 kV (positive or negative polarity) and the resulting current ranges from about 5 microamps to about 40 microamps.

The nozzle apparatus is preferably placed about 2 centimeters to about 20 centimeters away from the surface of the device to be coated. Furthermore, more than one nozzle apparatus can be used at the same time for the improved process of the invention. A rotating carousel can be used for large devices, or when coating on a manufacturing scale.

In another embodiment, the coating material is a dry powder formulation. Powder coating is accomplished as described, for example, in U.S. Pat. No. 5,695,826. If the powder is agglomerated, it can be first deagglomerated and entrained in air as described in U.S. Pat. No. 5,035,364. In the case of polymer powders, a short heating step can be added to heat the polymer powder to a temperature sufficient to cause the powder to melt and flow, and possibly increase adherence to the medical device. In the case of monomer powders, heat or ultraviolet (UV) radiation may be used to polymerize, or cure, the monomer on the device.

Non-conductive medical devices of the present invention include but are not limited to tissue engineering scaffolds, such as non-woven felts, lyophilized foams, or woven meshes, and complex-shaped medical devices, such as suture anchors, sutures, staples, surgical tacks, clips, plates, screws, and the like. A complex-shaped medical device is defined as any device that may have edges, recesses, depressions, cavities, channels, curves, and sharp sides, and may not be symmetric.

Non-conductive medical devices suitable for the present invention are biocompatible and preferably fabricated using biodegradable polymers. Biodegradable polymers readily break down into small segments when exposed to moist body tissue or physiological enzymes. The segments are either absorbed by the body or passed by the body. More particularly, the biodegraded segments do not elicit permanent chronic foreign body reaction, because they are absorbed by the body or passed from the body such that no permanent trace or residual amount of the segment is retained by the body. Suitable biodegradable polymers for use in complex-shaped medical devices include without limitation homopolymers such as poly(glycolide), poly(lactide), poly(epsilon-caprolactone), poly(trimethylene carbonate) and poly(para-dioxanone); and copolymers, such as poly(lactide-co-glycolide), or PLGA, poly(epsilon-caprolactone-co-glycolide, and poly(glycolide-co-trimethylene carbonate). The polymers may be statistically random copolymers, segmented copolymers, block copolymers or graft copolymers. Other biodegradable polymers include albumin; casein; waxes such as fatty acid esters of glycerol; glycerol monostearate and glycerol distearate; starch, crosslinked starch; simple sugars such as glucose and polysucrose; polyvinyl alcohol; gelatine; hyaluronic acid; modified celluloses such as, hydroxypropyl cellulose, hydroxypropyl-ethyl cellulose, hydroxypropyl-methyl cellulose, sodium carboxymethyl cellulose, and cellulose acetate; sodium alginate; polymaleic anhydride esters; polyortho esters;

polyethyleneimine; glycols such as polyethylene glycol, methoxypolyethylene glycol, and ethoxypolyethylene glycol; polyethylene oxide; poly(1,3-bis-p-carboxyphenoxy propane-co-sebacic anhydride); N,N-diethylaminoacetate; and block copolymers of polyoxyethylene and polyoxypropylene; and combinations thereof.

The polymers useful for forming the coatings on the surface of the medical device should be biocompatible, and are preferably biodegradable. The biodegradable polymers listed above as possible candidates for use in fabricating the medical device may also be used as coating polymers for the improved process of this invention.

Solvents suitable for forming the liquid coating formulations are ones that can dissolve the polymeric material into solutions or form dispersions of the polymeric materials in the solvent. Any solvent that does not alter or adversely impact the medical device can be employed. Preferably, the solvents are polar solvents, although non-polar solvents may also be used. Examples of useful solvents include chloroform, methylene chloride, ethyl acetate, acetone, isopropanol, and ethyl alcohol. The amount of polymeric material in the coating formulation should range from about 1 to about 60 percent weight/weight (w/w). Preferably, the amount of polymer in the coating formulation should be from about 1 to about 20 percent. The suitable viscosities of the coating solution range from about 1 centipoise (cps) to about 500,00 cps.

Coating formulations useful for the improved process of the present invention may also include a biologically active material. The terms “biologically active material” or “bioactive material” encompass therapeutic agents such as drugs and also genetic materials and biological materials. Suitable genetic materials include DNA or RNA such as, without limitation, DNA/RNA encoding a useful protein and DNA/RNA intended to be inserted into a human body including viral vectors and non-viral vectors. Suitable biological materials include cells, cell trophic factors, cell lysates, cell conditioned media, yeasts, bacteria, proteins, peptide, cytokines and hormones. Examples of suitable peptides and proteins include growth factors such as GDF-5, VegF, FGF-2, FGF-1, bone morphogenic proteins, BMP-2, BMP-3, BMP-4, BMP-5, BMP-6, BMP-7, BMP-8, BMP-9, BMP-10, BMP-11, BMP-12, BMP-13, BMP-14, BMP-15, BMP-16. These proteins can be provided as homodimers, heterodimers, or combinations thereof, alone or together with other molecules. Cells can be of human origin (autologous or allogeneic) from an animal source (xenogeneic), or genetically engineered, if desired to deliver proteins of interest at the transplant site. Cells include whole bone marrow, bone marrow derived mono-nuclear cells, progenitor cells, stem cells, pluripotent stem cells, and fibroblasts.

Biologically active materials also include, without limitation, antiinfectives, such as antibiotics and antiviral agents; analgesics and analgesic combinations; anorexics; antihelmintics; antiarthritics; antiasthmatic agents; anticonvulsants; antidepressants; antidiuretic agents; antidiarrheals; antihistamines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators, including general coronary, peripheral and cerebral; central nervous system stimulants; hormones, such as estradiol and other steroids, including corticosteroids; hypnotics; immunosuppressives; muscle relaxants; parasympatholytics; psychostimulants; sedatives; tranquilizers; naturally derived or genetically engineered proteins, polysaccharides, glycoproteins, or lipoproteins; oligonucleotides, antibodies, antigens, cholinergics, chemotherapeutics, hemostatics, clot dissolving agents, radioactive agents and cystostatics.

If the coating formulation is a polymeric solution, the bioactive material can be mixed into the solution. In this way the polymer and bioactive material are co-deposited. Or, in a multi-step process, a solution of bioactive can first be coated on the device, followed by a coating of polymer solution.

If the coating material is a dry polymer powder, a bioactive material can be encapsulated in the polymer powder to form microparticles. The bioactive-loaded microparticles can be deposited onto the biodegradable polymeric substrate. Alternatively, microparticles of polymer can be mixed with microparticles of bioactive and co-deposited. Or, in a multi-step process, microparticles of bioactive material can be coated on the substrate, followed by a coating of polymer microparticles. Various combinations of solution and powder coating deposition including but not limited to multi-step layered coatings of solution-solution, powder-solution, and powder-powder coatings can be accomplished with the coating process described herein.

The following comparative example illustrates the poor results obtained when the prior art electrostatic coating process is used to attempt applying a biocompatible coating to a tissue engineering scaffold.

### COMPARATIVE EXAMPLE 1

### Electrostatic Coating of a Non-woven Scaffold Without Pre-treatment with a Polar Liquid to Induce a Temporary Conductive Layer

A dry lay non-woven needle punched felt scaffold was made of 10/90 mole ratio poly(lactide-co-glycolide) (10/90 PLA/PGA) fibers (Ethicon, Inc., Somerville, N.J.). The non-woven scaffold had a nominal density of 108 milligrams per cubic centimeter, and a thickness of 2.14 millimeters. The scaffold was scoured to remove finishing substances by soaking and rinsing in isopropanol and water respectively. The rinsed scaffold was dried under flowing nitrogen and subsequently vacuum dried. When dry, the sheet was cut into 2.54 by 2.54 square centimeter samples. The dry sample was placed on a stainless steel frame 6 centimeters from the spray nozzle of an electrohydrodynamic nozzle and grounded. The electrohydrodynamic nozzle apparatus used is commercially available from Terronics Development Corporation (Elwood, Ind.).

A coating formulation containing 5 percent weight/volume (w/v) solution of 50/50 mole ratio poly(lactide-co-glycolide)(50/50 PLA/PGA) in ethyl acetate was prepared and placed into the electrohydrodynamic nozzle apparatus. The inherent viscosity, or I.V., of the 50/50 PLA/PGA was measured using a 50 bore Cannon-Ubbelhode dilution viscometer immersed in a thermostatically controlled water bath at 30° C. utilizing Hexafluoroisopropanol (HFIP) as the solvent at a concentration of 0.1 gram/deciliter. The I.V of the polymer was 0.61 deciliter/gram.

The coating formulation in the chamber of the apparatus was electrically charged using the voltage power source connected to the apparatus that was set at 8 kilovolts (negative polarity). The flow rate of the coating formulation at the nozzle opening was 2 milliliters per hour. The coating processing time was 6 minutes and the temperature of the processing environment was room temperature. The coating particles were observed being repelled by the scaffold and turning back towards the nozzle again.

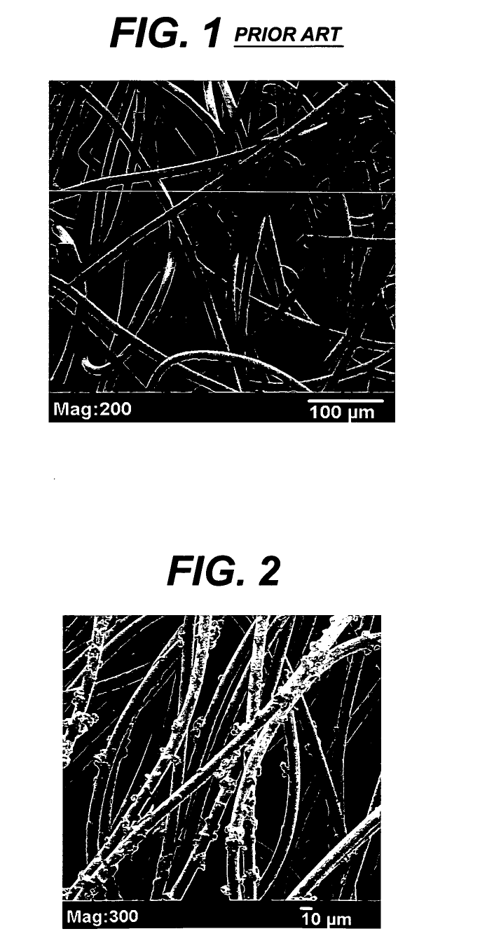

The surface of the non-woven felt scaffold was examined by a scanning electron microscope (Jeol JSM05900LV, Peabody, Mass.), and a micrograph of the surface is shown in FIG. 1. In this micrograph it can be observed that even after six minutes of processing no coating is apparent on the surface of the substrate.

The following examples are illustrative of the principles and practice of the present invention, although not limited thereto.

### EXAMPLE 1

### Electrostatic Coating of a Non-woven Scaffold with Pre-treatment with a Polar Liquid to Induce a Temporary Conductive Layer

In this example, Comparative Example 1 was substantially reproduced as detailed below, except that the pretreatment step of the improved process of this invention was added.

A dry lay non-woven needle punched felt scaffold was made of 10/90 mole ratio poly(lactide-co-glycolide) (10/90 PLA/PGA) fibers(Ethicon, Inc., Somerville, N.J.). The non-woven scaffold had a nominal density of 108 milligrams per cubic centimeter, and a thickness of 2.14 millimeters. The scaffold was scoured to remove finishing substances by soaking and rinsing in isopropanol and water respectively. The rinsed scaffold was dried under flowing nitrogen and subsequently vacuum dried. When dry, the sheet was cut into 2.54 by 2.54 square centimeter samples. A sample was pre-treated by dipping the sample into 99 percent isopropanol (Aldrich, St. Louis, Mo.). Excess ispropanol was shaken off the sample, which was then placed on a stainless steel frame 8 centimeters from the spray nozzle of an electrohydrodynamic nozzle apparatus and grounded. The electrohydrodynamic nozzle apparatus used is commercially available from Terronics Development Corporation (Elwood, Ind.).

A liquid coating formulation containing 5 percent weight/volume (w/v) solution of 50/50 mole ratio poly(lactide-co-glycolide)(50/50 PLA/PGA) in ethyl acetate was prepared and placed into the electrohydrodynamic nozzle apparatus. The inherent viscosity, or I.V., of the 50/50 PLA/PGA was measured using a 50 bore Cannon-Ubbelhode dilution viscometer immersed in a thermostatically controlled water bath at 30° C. utilizing Hexafluoroisopropanol (HFIP) as the solvent at a concentration of 0.1 gram/deciliter. The I.V of the polymer was 0.61 deciliter/gram. The liquid coating formulation in the chamber of the apparatus was electrically charged using the voltage power source connected to the apparatus that was set at 8.5 kilovolts (negative polarity). The flow rate of the coating formulation at the nozzle opening was 1 milliliter per hour. The coating processing time was 30 seconds and the temperature of the processing environment was room temperature. The surface of the non-woven felt scaffold was examined by a scanning electron microscope (Jeol JSM05900LV, Peabody, Mass.), and a micrograph of the surface is shown in FIG. 2. In contrast to the results achieved in Comparative Example 1, the coating is apparent on the surface of scaffold as “beads” that adhere to the fibers of the scaffold.

### EXAMPLE 2

### Electrostatic Coating of an Injection Molded Screw with a Liquid Coating Formulation

An injection molded screw, approximately 3 centimeters in length, composed of 85 percent weight/weight (w/w) poly(lactic acid) and 15 percent (w/w) tri-calcium phosphate was pre-treated by dipping the screw into 99 percent isopropanol (Aldrich, St. Louis, Mo.), fixed onto a metal sample holder, grounded, and placed 6 centimeters from the spray nozzle of an electrohydrodynamic nozzle apparatus (commercially available from Terronics Development Corporation, Elwood, Ind.). An inductor ring was placed in close proximity of the nozzle tip. A liquid coating formulation solution containing 20 percent weight/volume (w/v) of 50/50 mole ratio poly(lactide-glycolide) in ethyl acetate was prepared and placed into the electrohydrodynamic nozzle apparatus. The inherent viscosity, or I.V., of the 50/50 PLA/PGA was measured using a 50 bore Cannon-Ubbelhode dilution viscometer immersed in a thermostatically controlled water bath at 30 degrees Celsius utilizing hexafluoroisopropanol (HFIP) as the solvent at a concentration of 0.1 gram/deciliter. The I.V. of the polymer was 0.61 deciliter/gram. The liquid coating formulation in the chamber of the apparatus was electrically charged using the voltage power source connected to the apparatus that was set at 6 kilovolts (negative polarity). The flow rate of the coating formulation at the nozzle opening was 3 milliliters per hour. The coating processing time was 3 minutes and the temperature of the processing environment was 20 degrees Celsius. Device was air-dried.

The coated screw was examined by an optical microscope. The coating completely covers the surface of the bone screw but does not mask the threads of the screw necessary for its placement in the physiological environment.

### EXAMPLE 3

### Electrostatic Coating of an Injection Molded Screw with a Dry Powder Coating Formulation

Poly(monostearoyl glycerol-co-succinate), or MGSA powder was synthesized as follows, 2510 grams of monostearoyl glyceride (Van Waters & Rogers, Quest International, Hoffman Estates, Ill.), 770.4 grams of succinic anhydride (Acros Organics via Fisher Scientific, Morris Plains, N.J.), and 1.41 milliliters of 0.33 molar stannous octoate in toluene (Ethicon, Inc., Comelia, Ga.) were placed in an 8 CV Helicone Mixer (manufactured by Design Integrated Technology, Inc. of Warrenton, Va.). During the initial five hours of mixing a vacuum was applied. Contents of mixer were stirred at a rate of initially 8 rpm and increased to 20 rpm. Reaction time was approximately 46.5 hours. A Mettler-Toledo FP62 melting apparatus was used to measure the melting point of the polymer and it was found to be 50.4 degrees Celsius. Molecular weight was determined by gel permeation chromatography (Waters Corporation, Milford, Mass.) in tetrahydrofuran with polystyrene standards. The weight average molecular weight was found to be 38,489 daltons.

The powder was converted to polymer microparticles on a rotating disk apparatus. The powder first melted and equilibrated to 110 degrees Celsius and fed at a controlled rate of 3.5 grams/second to the center of a 3 inch rotary disk that was run at 7500 rpm. The disk surface was heated using an induction heating mechanism to 130 degrees Celsius to ensure that the polymer was in a liquid state on the surface of the disk. The rotation of the disk caused a thin liquid film of polymer to be formed on the surface of the disk. The liquid film was thrown radially outward from the surface of the disk and droplets solidified upon contact with air in the rotating disk apparatus chamber to form polymer microparticles. The solid microparticles were then collected using a cyclone separator. Microspheres were cryo-sieved and only the fraction of particles less than 53 microns was used.

An injection molded screw, approximately 3 centimeters in length, composed of 85 percent weight/weight (w/w) poly(lactic acid) and 15 percent (w/w) tri-calcium phosphate was pre-treated by dipping the screw into 99 percent isopropanol (Aldrich, St. Louis, Mo.), fixed onto a metal sample holder, and grounded. The microspheres were fed into the injector of a powder coater (commercially available from Terronics Development Corporation, Elwood, Ind.) where it was mixed with air, accelerated, and gently dispersed and injected as a uniformly dispersed powder cloud that was electrostatically charged via an enhanced negative corona. A vibrator feeder (Quaver-ACI, Cole-Palmer, Vernon Hills, Ill.) was used to feed the powder as evenly as possible to the injector, where it was mixed with air. The powder/air mixture flowed at 20 SCFM and the voltage used to charge the powder particles was 16 kV (negative polarity). The electrostatically charged powder cloud coated the screw. The temperature of the coating environment was 25 degrees Celsius. The coated screw was placed in a convection oven set at 54 degrees Celsius for 10 seconds following electrostatic coating to melt the coating powder sufficiently to flow. The coated polymeric device was examined by an optical microscope. The melted microsphere coating was observed on the complex surface of the bone screw and does not mask the threads of the screw necessary for its placement in the physiological environment.

We claim:

1. An improved electrostatic coating process for coating a medical device of the type wherein a medical device is provided, the medical device is placed on a metallic support, the supported medical device is grounded, and the surface of the supported medical device is electrostatically coated with a coating formulation; wherein the surface of the medical device is a non-conductive surface, and the improvement comprises the step of inducing a temporary conductive layer to the non-conductive surface of the medical device prior to the step of electrostatically coating the surface of the medical device with the coating formulation.

2. The improved electrostatic coating process of claim 1 wherein the step of inducing the temporary conductive layer is carried out using a polar liquid.

3. The improved electrostatic coating process of claim 2 wherein the polar liquid is selected from the group consisting of tetrahydrofuran, acetone, ethyl acetate, N-methylpyrrolidone, dimethyl sulfoxide, isopropanol, ethyl alcohol, methylene chloride, and methyl ethyl ketone.

4. The improved electrostatic coating process of claim 3 wherein the polar liquid is ethyl acetate or isopropanol.

5. The improved electrostatic coating process of claim 2 wherein the polar liquid is either dipped into or sprayed onto the surface of the medical device to induce the temporary conductive layer.

6. The improved electrostatic coating process of claim 5 wherein the medical device is a tissue engineering scaffold or a complex-shaped medical device.

7. The improved electrostatic coating process of claim 6 wherein the tissue engineering scaffold is a non-woven felt, a woven mesh, or a foam.

8. The improved electrostatic coating process of claim 6 wherein the complex-shaped medical device is a suture anchor, suture, staple, surgical tack, clip, plate, or screw.

9. The improved electrostatic coating process of claim 5 wherein the medical device is composed of a biodegradable polymer selected from the group consisting of poly(glycolide), poly(lactide), poly(epsilon-caprolactone), poly(trimethylene carbonate), poly(para-dioxanone), poly(lactide-co-glycolide), poly(epsilon-caprolactone-co-glycolide, poly(glycolide-co-trimethylene carbonate), albumin, casein, fatty acid esters of glycerol, glycerol monostearate, glycerol distearate, starch, crosslinked starch, glucose, polysucrose, polyvinyl alcohol, gelatine, hyaluronic acid, hydroxypropyl cellulose, hydroxypropyl-ethyl cellulose, hydroxypropyl-methyl cellulose, sodium carboxyymethyl cellulose, cellulose acetate, sodium alginate, polymaleic anhydride esters, polyortho esters, polyethyleneimine, polyethylene glycol, methoxypolyethylene glycol, ethoxypolyethylene glycol, polyethylene oxide; poly(1,3 bis(p-carboxyphenoxy), poly(1,3-bis-p-carboxyphenoxy propane-co-sebacic anhydride), N,N-diethylaminoacetate, and block copolymers of polyoxyethylene and polyoxypropylene.

10. The improved electrostatic coating process of claim 5 wherein the coating formulation is a liquid formulation or a dry powder formulation.

11. The improved electrostatic coating process of claim 10 wherein the liquid formulation is a polymer in a solvent, an emulsion, or a suspension.

12. The improved electrostatic coating process of claim 10 wherein the dry powder formulation is a monomer powder or a polymer powder.

13. The improved electrostatic coating process of claim 5 wherein the coating formulation is a biodegradable polymer.

14. The improved electrostatic coating process of claim 5 wherein the coating formulation contains a biologically active material.

15. A non-conductive medical device coated by the improved process of claim 1.

16. A non-conductive medical device coated by the improved process of claim 5.
